(19) United States
(12) Patent Application Publication
(10) Pub. No.: US 2009/0000027 A1
(43) Pub. Date: Jan. 1, 2009

(54) STRETCHER PADS FOR USE WITH STRETCHERS

(75) Inventor: Ben W. Jarrett, III, Maryville, IL (US)

Correspondence Address: HARNESS, DICKEY, & PIERCE, P.L.C, 7700 Bonhomme, Suite 400, ST. LOUIS, MO, 63105, US

(73) Assignee: GRADIENT PRESSURE PRODUCTS, LLC, St. Louis, MO (US)

(21) Appl. No.: 11/770,673

(22) Filed: Jun. 28, 2007

(51) Int. Cl. A61F 5/32; A61G 1/00; A61G 7/08

(52) U.S. Cl. 5/86.1; 5/625

(57) ABSTRACT

A stretcher pad for use with a stretcher in transporting a body from one location to at least another location in a generally horizontal position. The stretcher pad may improve pressure distribution to the body being transported by the stretcher. The stretcher generally comprises a wheeled frame for rolling the stretcher over a surface and transporting the body from one location to at least another location. A generally elongate pad is supported by the wheeled frame for receiving the body in a generally horizontal position on the stretcher. A pressure management insert is disposed at least partly within the pad for supporting at least part of a trunk of the body on the stretcher.

### FIELD

The present disclosure relates generally to pads for use with stretchers, and more particularly to a pad for use with a stretcher having a pressure management insert within the pad for improving support to at least part of a patient's body.

### BACKGROUND

The statements in this section merely provide background information related to the present disclosure and may not constitute prior art.

A stretcher (or gurney) is often used to transport a patient between locations. For example, a stretcher may be used to transport a patient from an ambulance to a hospital, between rooms within a hospital, etc. Generally, a pad is provided on an upper surface of the stretcher so that the patient may be comfortably supported on the pad while being transported.

In some situations a patient may remain on a stretcher for long periods of time, for example up to twenty-four hours, after being transferred to the stretcher (and sometimes after being subsequently transported by the stretcher). In these situations, portions of the patient's body in contact with the stretcher pad (e.g., a trunk region of the patient's body, etc.) may be subjected to continuous, unrelieved pressures from the pad. These unrelieved pressures may reduce blood flow to the surrounding soft tissue in the affected areas. Consequently, the tissue may break down and form pressure ulcers, which if left untreated can become dangerous and difficult to cure.

### SUMMARY

According to various aspects of the present disclosure, exemplary embodiments are provided of stretchers and stretcher pads for use with stretchers in transporting bodies from one location to at least another location in a generally horizontal position. The stretcher pads used with the stretchers may improve pressure distribution to the bodies on the stretchers. In one exemplary embodiment, a stretcher generally comprises a wheeled frame for rolling the stretcher over a surface and transporting a body from one location to at least another location. A generally elongate pad is supported by the wheeled frame for receiving the body in a generally horizontal position on the stretcher. A pressure management insert is disposed at least partly within the pad for supporting at least part of a trunk of the body on the stretcher.

In another exemplary embodiment, a stretcher may be used in transporting a body from one location to at least another location in a generally horizontal position with improved pressure distribution to the body. The stretcher generally comprises a generally elongate foam pad and a frame for supporting the foam pad. A pressure management insert is disposed at least partly within the foam pad for supporting at least part of a trunk of the body received on the pad. A longitudinal length dimension of the inflatable pressure management insert is less than or equal to about one-half of a longitudinal length dimension of the pad.

Other aspects of the present disclosure relate to methods for transporting a body in a generally horizontal position from one location to at least another location. In one exemplary embodiment, a method generally includes positioning the body in a generally horizontal position on a generally elongate foam pad supported by a frame. The positioning generally includes supporting at least part of the body with the pad so that one or more extremities of the body are on the pad, and supporting at least part of a trunk of the body with a pressure management insert within the pad, whereby the pressure management insert substantially uniformly distributes pressure along said at least part of the trunk of the body.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a perspective view of an exemplary embodiment of a stretcher pad for use with a stretcher in transporting a body;

FIG. 2 is a perspective view of the stretcher pad of FIG. 1;

FIG. 3 is an exploded perspective view of the stretcher pad of FIG. 2;

FIG. 4. is a top view of the stretcher pad of FIG. 2;

FIG. 5 is a section view of the stretcher pad of FIG. 4 taken in a plane including line 5-5 in FIG. 4;

FIG. 6 is a section view of the stretcher pad of FIG. 4 taken in a plane including line 6-6 in FIG. 4;

FIG. 7 is a graphic view illustrating exemplary pressure distribution over the stretcher pad of FIG. 1 when a body is positioned on the pad; and

FIG. 8 is perspective view of another exemplary embodiment of a stretcher pad in which the pad is received within a protective cover.

### DETAILED DESCRIPTION

The following description is merely exemplary in nature and is not intended to limit the present disclosure, application, or uses. It should be understood that throughout the drawings, corresponding reference numerals indicate like or corresponding parts and features.

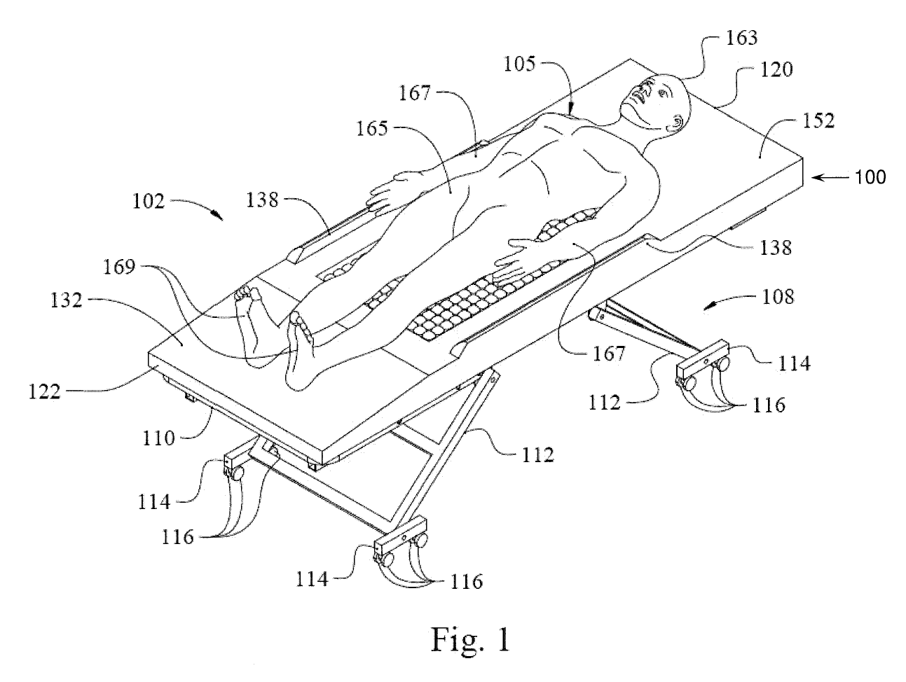

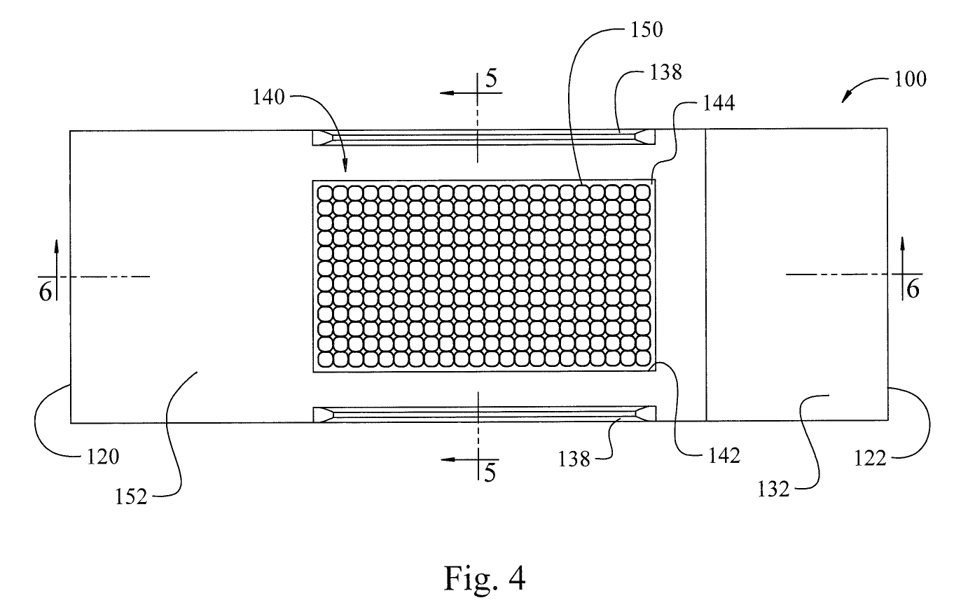

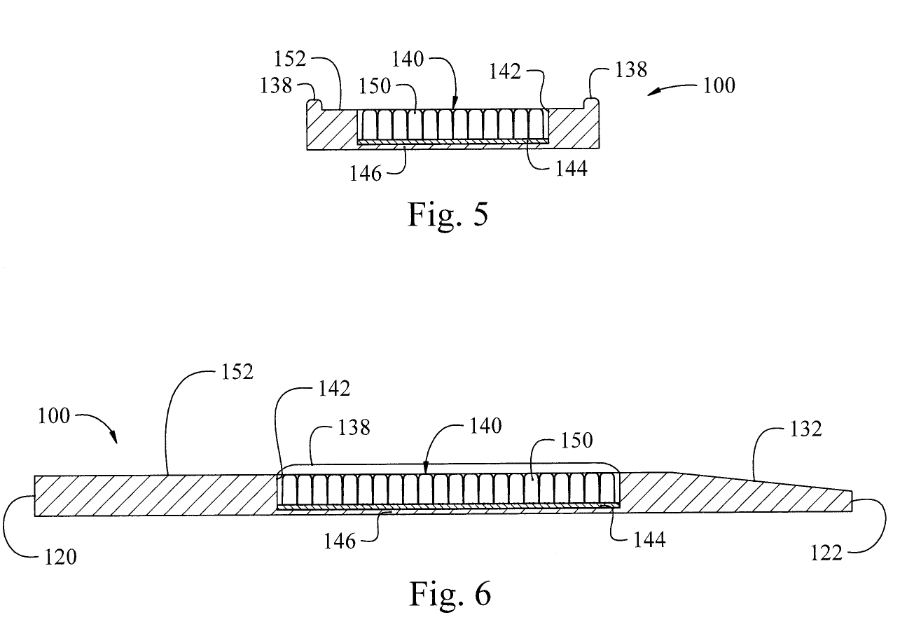

With reference now to the drawings, FIGS. 1-6 illustrate an exemplary embodiment of a stretcher pad 100 for use with a stretcher (or gurney) 102 (FIG. 1) in transporting a body 105 (FIG. 1) from one location to at least another location. The stretcher pad 100 may provide improved pressure distribution to the body 105 on the stretcher 102, which may be used, for example, to transport the body 105 from an emergency transport vehicle (e.g., an ambulance, etc.) to a medical care facility (e.g., a hospital, etc.), between rooms within the medical care facility, etc. As used herein, a body may include a patient's body, including, for example, a person's body, an animal's body, etc.

With reference to FIG. 1, the stretcher 102 is a generally lightweight device configured to be rolled along a ground surface to transport the patient's body 105. The stretcher 102 includes a frame 108 having an upper surface 110 upon which the stretcher pad 100 is supported, and a pair of legs 112 extending generally downwardly from the upper surface 110 for holding the upper surface and stretcher pad 100 above the ground surface. Rollers 114 are attached to end portions of the legs 112, with two rollers 114 attached to each leg. Each roller 114 includes four wheels 116 which allow the stretcher 102 to be rolled over the ground surface. The legs 112 of the frame 108 are pivotally connected to the upper surface 110 of the frame such that the legs 112 may be pivoted upwardly toward the upper surface 110 to collapse the frame 108 to a generally horizontal position when desired. It is understood that the frame 108 may be configured differently than described and illustrated herein within the scope of the present disclosure. For example, in one exemplary embodiment a stretcher includes a frame with a single central leg post extending generally downwardly from an upper surface, and a wheeled base attached to the central leg post for allowing rolling movement of the stretcher over a ground surface. The wheeled base may include, for example, six wheels, including two wheels (e.g., one wheel on each side of the wheeled base) of increased size/diameter to help facilitate improved movement and/or stabilization of the stretcher.

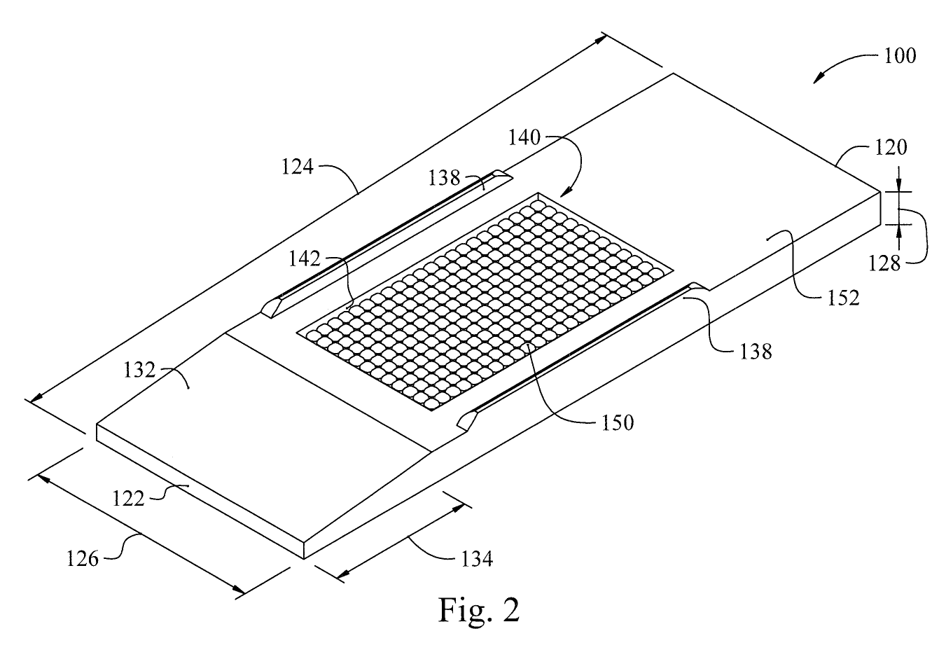

With reference now to FIG. 2, the stretcher pad 100 is generally thin and elongate in shape with first and second opposing longitudinal ends 120 and 122. A length dimension 124 of the illustrated pad 100 is about seventy-five inches, a width dimension 126 of the illustrated pad 100 is about twenty-nine inches, and a thickness dimension 128 of the illustrated pad 100 is generally about four inches (toward the first longitudinal end 120). In other exemplary embodiments, stretcher pads may include length dimensions less than about eighty inches, width dimensions less than about thirty-six inches, and thickness dimensions less than about five inches. In addition, the illustrated stretcher pad 100 is constructed from a foam material (e.g., therapeutic foam, etc.) that may provide durability to the pad, as well as comfort and therapeutic support to a patient's body lying on the pad 100. Moreover, at least part of the illustrated pad 100 may be constructed from layers of foam material to improve therapeutic operation and function of the pad 100. For example, at least part of the pad 100 toward the first longitudinal end 120 may be constructed from three layers (or regions) of foam material, each layer having a different firmness (or density) so that the layers of foam material increase in firmness (softer to firmer) from an upper layer (e.g., adjacent the patient's body 105 (FIG. 1)) to a lower layer (e.g., adjacent the upper surface 110 of the stretcher frame 108 (FIG. 1)). These layers may improve support to the patient's body 105 and may allow the pad 100 to accommodate heavier body weights. In other exemplary embodiments, stretcher pads may include, for example, one or more layers of foam material. In still other exemplary embodiments, a stretcher pad may be constructed from nylon, etc. in a baffled configuration, etc.

The stretcher pad 100 includes a sloped heel portion 132 toward the second longitudinal end 122 of the pad 100. The sloped heel portion 132 extends a heel length dimension 134 along the illustrated pad 100 of about eighteen inches, up to the second longitudinal end 122 of the pad, such that the thickness dimension 128 of the pad 100 at the second longitudinal end 122 is reduced to about two inches. The sloped heel portion 132 may be constructed, for example, from a foam material separate and/or different from the above-described layers of foam material located toward the first longitudinal end 120 of the pad 100. For example, the foam material of the sloped heel portion 132 may have a lower firmness (e.g., may be softer) than the other layers of foam material of the pad 100. However, firmer foam materials may be used for the sloped heel portion 132 within the scope of the present disclosure. In addition, the layers of foam material located toward the first longitudinal end 120 of the pad 100 may also form at least part of the sloped heel portion 132 within the scope of the present disclosure. In other exemplary embodiments, stretcher pads may include sloped heel portions having heel length dimensions larger than or smaller than about eighteen inches. In still other exemplary embodiments, stretcher pads may include sloped heel portions extending up to longitudinal ends of the pads such that thickness dimensions of the pads' longitudinal ends are larger than or smaller than about two inches. In further exemplary embodiments, stretcher pads may not include sloped heel portions such that thickness dimensions of the pads are generally uniform from first longitudinal ends of the pads to second longitudinal ends of the pads.

In the illustrated stretcher pad 100, raised side bolsters 138 extend generally longitudinally along a portion of the longitudinal side edges of the stretcher pad 100. The side bolsters 138 provide a raised structure above the stretcher pad 100 at the side edges to at least partly retain the patient's body 105 on the stretcher 102 against rolling or sliding off the pad 100, for example during transportation. In the illustrated stretcher pad 100, the raised side bolsters 138 are generally formed monolithically with the stretcher pad 100. In other exemplary embodiments, stretcher pads may include raised side bolters formed separately from the pads and attached thereto. In still other exemplary embodiments, stretcher pads may include raised side bolters extending substantially along longitudinal side edges of the stretcher pads and/or at least partly around a perimeter of the stretcher pads (e.g., at least partly around longitudinal ends of the stretcher pads, etc.). In still further exemplary embodiments, stretcher pads may be free of raised side bolters.

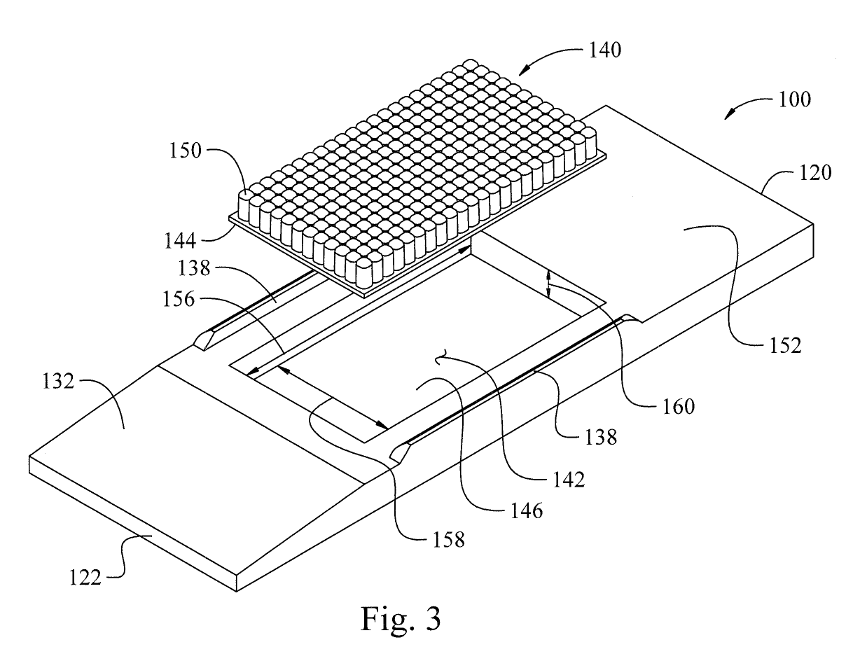

With additional reference to FIGS. 3-6, the stretcher pad 100 includes a pressure management insert 140 disposed in the stretcher pad 100 within an opening 142 formed in the pad 100. The pad opening 142 may be formed (e.g., cut into the pad 100, etc.) after the pad 100 is formed, or it may be formed (e.g., molded into the pad 100, etc.) at the same time the pad 100 is being formed.

The pressure management insert 140 and the pad opening 142 are generally similarly sized so that the insert 140 is substantially completely received within the opening 142. A base 144 of the pressure management insert 140 is positioned generally over a floor 146 of the pad opening 142, and fluid-retaining cells 150 (broadly, chambers) mounted on the insert's base 144 are received within the pad opening 142 so that tops of the cells 150 are generally flush with an upper surface 152 of the pad 100. The pressure management insert 140 may be releasably retained within the pad opening 142 by hook and loop fasteners (e.g., Velcro®, etc.). For example, the pressure management insert 140 may include hook fasteners and the stretcher pad 100 may include loop fasteners within the pad opening 142 for receiving the hook fasteners and securing the insert 140 within the pad opening 142. The insert 140 may be removed from the pad 100 by, for example, disengaging the hook and loop fasteners and pulling the insert 140 out of the opening 142 (e.g., for cleaning, repair, etc.). In other exemplary embodiments, stretcher pads may include pressure management inserts releasably retained within openings in the stretcher pads by, for example, friction fit, snap connectors, other releasable connectors, etc. In still other exemplary embodiments, stretcher pads may include pressure management inserts fixedly retained within openings in the stretcher pads by, for example, adhesive, hot melt processes, etc. In yet other exemplary embodiments, stretcher pads may include pressure management inserts retained within stretcher pad openings extending through the pads such that the pads are free of floors at the openings. In further exemplary embodiments, stretcher pads may include pressure management inserts received within openings in the stretcher pads in which the inserts are substantially larger than or smaller than the pad openings.

The opening 142 of the stretcher pad 100 (and thus the pressure management insert 140 of the pad received within the pad opening 142) is located about eighteen inches inwardly from the first longitudinal end 120 of the illustrated pad 100, and about five inches inwardly from each longitudinal side edge. In addition, the pad opening 142 (and thus the pressure management insert 140) includes a length dimension 156 of about thirty-four inches, a width dimension 158 of about eighteen inches, and a thickness dimension 160 of about three and one-half inches. In other exemplary embodiments, stretcher pads may include pressure management inserts disposed within openings in the pads located more than or less than eighteen inches from longitudinal ends of the pads and/or more than or less than five inches from side edges of the pads. In still other exemplary embodiments, stretcher pads may include pressure management inserts (and thus pad openings) with length dimensions less than about forty inches, width dimensions less than about twenty inches, and thickness dimensions less than about four inches.

It should now be appreciated that the stretcher pad 100 has an upper facing surface area of about 2,113 square inches (excluding the surface area of the pad opening 142), and the pressure management insert 140 has an upper facing surface area of about 612 square inches. Therefore in the illustrated stretcher pad 100, the value of the upper facing surface area of the pressure management insert 140 is about three-tenths the value of the upper facing surface area of the pad 100. In other exemplary embodiments, stretcher pads may include pressure management inserts in which values of upper facing surface areas of the pressure management inserts and of the stretcher pads may be larger than or smaller than those disclosed herein. For example in one exemplary embodiment, the value of the upper facing surface area of the pressure management insert is about one-half or less the value of the upper facing surface area of the stretcher pad. In another exemplary embodiment, the value of the upper facing surface area of the pressure management insert is about two-fifths or less the value of the upper facing surface area of the stretcher pad.

As previously stated, the pressure management insert 140 includes multiple inflatable fluid-retaining cells 150 supported on the base 144 of the insert 140. The cells 150 are each generally tubular in shape and are configured to receive fluid into the cells 150 to generally inflate the insert 140 for supporting a portion of the patient's body 105 (FIG. 1). Inflating the cells 150 may be accomplished by introducing suitable fluid into the cells 150, including liquids (e.g., water, etc.), gases (e.g., air, helium, oxygen, etc.), or combinations thereof, from a suitable fluid source (e.g., an air compressor, etc.). In the illustrated embodiment, inflating the cells 150 may be accomplished by introducing air into the cells 150. The fluid retaining cells 150 are typically inflated prior to positioning the insert 140 within the opening 142 in the stretcher pad 100. However, the cells 150 may be inflated after positioning the insert 140 within the pad opening 142 and/or, for example, after positioning the patient's body 105 on the pad 100. In addition, the cells 150 may be formed from materials including neoprene, polyvinyl, etc. suitable for retaining fluid.

In the illustrated stretcher pad 100, the fluid-retaining cells 150 of the pressure management insert 140 are interconnected by fluid channels or passageways (not visible) so that fluid may be introduced into one cell 150 to thereby collectively inflate each of the other cells 150. For example, a single inlet may be provided in one of the cells 150 for receiving fluid into the cell for inflating all of the fluidly connected cells 150. A plug or other suitable structure (e.g., a fluid valve, etc.) may be included with the inlet to block or close the inlet once the cells 150 are filled with the desired amount of fluid. Valve systems may also be provided in the fluid channels between the cells 150 for controlling fluid flow between the cells 150. With this construction, the interconnected cells 150 may substantially evenly distribute fluid pressure within the interconnected cells. In other exemplary embodiments, stretcher pads may include pressure management inserts in which one or more individual cells include an inlet for receiving fluid into the cells for inflating the cells. In still other exemplary embodiments, stretcher pads may include pressure management inserts in which bases of the inserts include inlets, fluid channels, and valve assemblies for use in inflating fluid-retaining cells. In still other exemplary embodiments, stretcher pads may include pressure management inserts having inlets continuously connected to fluid sources for providing sufficient and continuous fluid flow through the inlets into cells of the inserts to inhibit deflation of the inserts. In further exemplary embodiments, stretcher pads may include pressure management inserts that may be deflated, or collapsed, when desired by releasing fluid from the inserts, for example through one or more inlets of the inserts. In still further exemplary embodiments, stretcher pads may include pressure management inserts with cells having outlets separate from inlets for deflating the inserts.

The illustrated pressure management insert 140 includes two hundred sixty-four tubular fluid-retaining cells 150. In other exemplary embodiments, stretcher pads may include pressure management inserts with more than or fewer than two hundred sixty-four fluid-retaining cells. For example, in one exemplary embodiment, a stretcher pad includes a pressure management insert with one hundred eighty cells. In another exemplary embodiment, a stretcher pad includes a pressure management insert with sixty fluid-retaining cells. In still other exemplary embodiments, stretcher pads may include pressure management inserts with fluid-retaining cells shaped other than tubular, including, for example, conical, cubical, non-cubical, etc.

With reference again to FIG. 1, use of the stretcher pad 100 to, for example, transport the patient's body 105 will be described. The patient's body 105 is positioned on the stretcher pad 100 on the upper surface 110 of the frame 108 of the stretcher 102 with the patient's body 105 oriented in a generally horizontal position facing generally upwardly. A head 163 of the patient's body is supported by the stretcher pad 100 generally toward the first longitudinal end 120 of the pad 100, a trunk portion 165 (including, for example, a hip region, etc.) of the patient's body 105 is at least partly supported over the pressure management insert 140 of the pad 100, arms 167 of the patient's body 105 are supported at least partly by the pad 100 adjacent the raised side bolsters 138, and feet 169 of the patient's body 105 are supported by the pad 100 over the sloped heel portion 132 toward the pad's second longitudinal end 122. The sloped heel portion 132 of the pad 100 positions the patient's feet 169 to decrease pressure on heels of the feet 136. It is to be understood that the patient's body 105 may be oriented differently on the stretcher 102 and stretcher pad 100 within the scope of the present disclosure. For example, the patient's body 105 may be oriented on the stretcher pad 100 facing generally downwardly.

Once the patient's body 105 is positioned on the stretcher pad 100, restraints (not shown) may be used to further secure the patient's body 105 on the stretcher 102 against rolling or sliding off the stretcher pad 100. For example, straps may be attached to the frame 108 for positioning over the patent's body 105 to further secure the patient's body on the stretcher 102. Once the patient's body 105 is securely positioned on the stretcher 102, the stretcher may be moved (e.g., rolled, etc.) over the ground surface to the desired location (e.g., a hospital, etc.). Once at the desired location, the patient's body may then be transferred off the stretcher 102, for example, to a hospital bed, or may be further transported to another location, for example a hospital operating room.

Unfortunately, however, after being positioned on the stretcher and before being transferred off the stretcher 102 or before being further transported to another location on the stretcher 102, the patient's body may remain on the stretcher 102 and stretcher pad 100 for a long period of time, for example, up to twenty-four hours. The stretcher pad 100 of the present disclosure may provide pressure relief to bodies of patients positioned on the stretcher 102 for long periods of time. The fluid-receiving cells 150 of the pressure management insert 140 immerse the patient's trunk portion 165 in the insert 140 and at least partially assume the shape of the immersed trunk portion 165. This increases surface area contact between the patient's trunk portion 165 and the insert 140 so that the individual cells 150 may provide substantially uniform support to the patient's trunk portion 165 immersed in the insert 140. In addition, pressures/forces from the cells 150 acting on the skin surface of the immersed trunk portion 165 may be reduced, thus improving blood flow to the immersed region and minimizing skin tissue break down and/or deformation. In addition, individual cells 150 of the pressure management insert 140 are generally spaced close enough together to engage and support each other when inflated and supporting portions of the patient's body 105. However, the individual cells 150 are moveable independent of the other adjacent cells 150 so that the cells may accommodate movement of the patent without rubbing skin of the patient's body 105. This provides a low friction and low shear environment when the patient moves to further minimize tissue break down and/or deformation. Moreover, the spaced cells 150 allow air to flow between the cells thus promoting a dry environment for the patient's body 105.

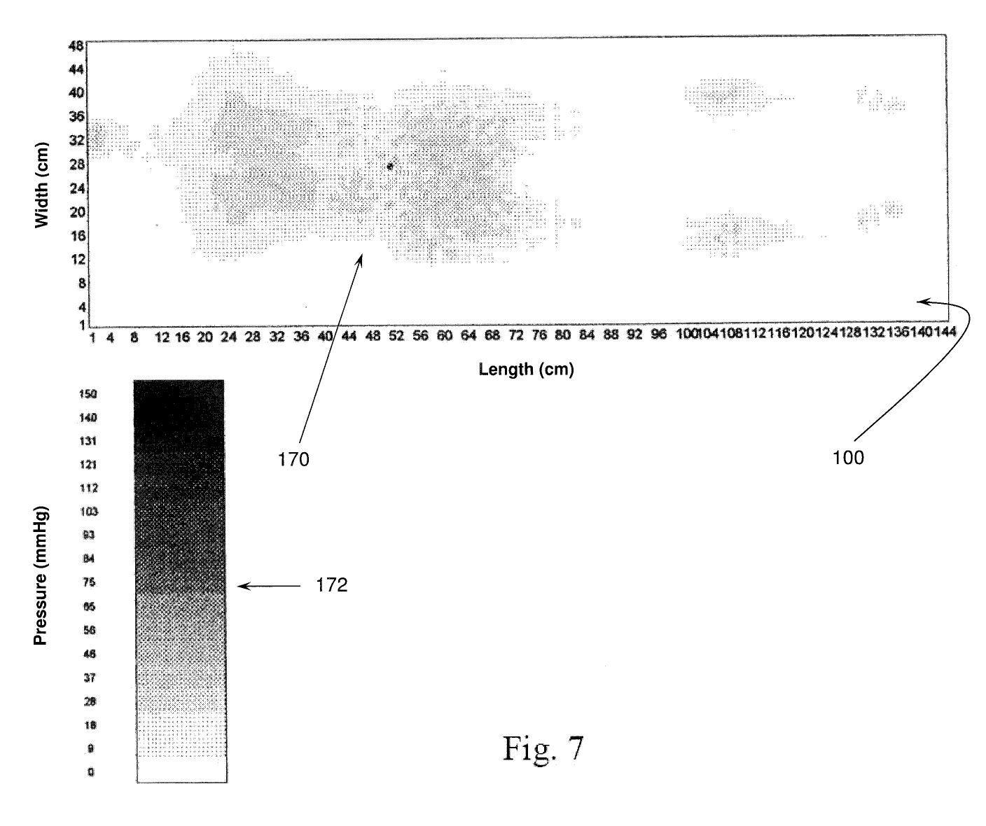

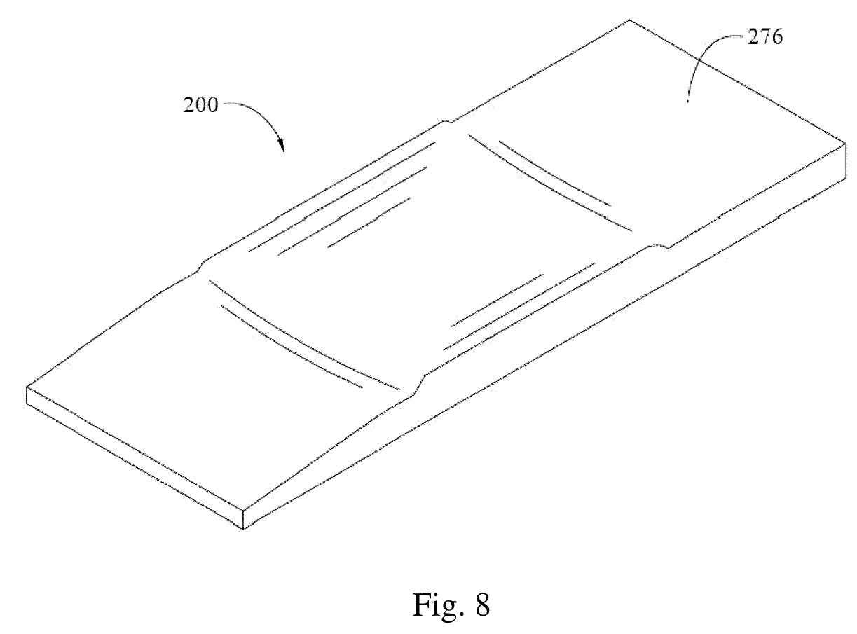

FIG. 7 illustrates exemplary pressure distribution 170 over a portion of the stretcher pad 100 when a patient's body is positioned on the pad 100. The pressure distribution 170 indicates pressure, in millimeters of mercury, at intervals of one square centimeter over the illustrated portion of the pad 100. Lighter shades of color in FIG. 7 indicate lower pressures, as described in legend 172. As can be seen in the illustrated portion of the pad 100, the pressure distribution 170 is generally uniform along a trunk portion of the patient's body immersed in the pressure management insert 140 of the pad 100. In FIG. 7, the pressure management insert 140 is located generally in the stretcher pad 100 between horizontal length dimensions of about 48 centimeters and about 82 centimeters. Pressures exerted on the patient's trunk portion by the pressure management insert 140 may range, for example, from about 15 millimeters of mercury to about 40 millimeters of mercury, and an average pressure may be about 25 millimeters of mercury.

It should now be appreciated that the stretcher pad 100 may include one or more zones (or regions) of support (e.g., therapy zones, etc.) for supporting different portions of the patient's body 105 on the stretcher pad 100. All of the patient's body 105 may thus be therapeutically supported, as desired and/or necessary, by the stretcher pad 100.

For example, the illustrated stretcher pad 100 may include three therapy zones. A first therapy zone may provide therapeutic support to, for example, the head 163 and arms 167 of the patient's body 105. This therapy zone may extend across the width 126 of the pad 100 and along the length 124 of the pad 100 from the first longitudinal end 120 of the pad 100 to the sloped heel portion 132 of the pad 100. As previously stated, this therapy zone may include, for example, three layers of foam material, with the upper layer (e.g., upper surface 152 adjacent the patient's body 105) being the softest layer and the lower layer (e.g., adjacent the upper surface 110 of the stretcher frame 108 and including at least part of the floor 146 of the pad opening 142) being the firmest layer. A central layer in this therapy zone (e.g., between the upper layer of foam material and the lower layer of foam material) may include, for example, a layer of foam material having a medium firmness. These layers may be of about equal thickness and may be generally uniformly oriented through the thickness 128 of the pad 100. However, the layers of foam material may have different firmnesses than described, and may be differently sized and differently oriented within the scope of the present disclosure. The heel portion 132 of the pad 100 may include a second therapy zone having foam material that is softer than each of the layers of foam material of the first therapy zone. The second therapy zone may extend across the width 126 of the pad 100 and along the length 134 of the sloped heel portion 132 of the pad 100. This second therapy zone may provide therapeutic support to the feet 169 (and heels) of the patient's body. Finally, the pressure management insert 140 of the pad 100 may include a third therapy zone providing therapeutic support to the trunk portion 165 of the patient's body 105. The stretcher pad 100 may include more than or fewer than three therapy zones within the scope of the present disclosure.

It should be understood that each exemplary therapy zone may, for example, provide a particular type of support to the portion of the patient's body over the zone. The desired support for each therapy zone may thus be predetermined (e.g., by material selection, etc.) based on the portion of the patient's body 105 to be supported. And different types of support may be provided to different portions of the patient's body 105. This unique combination of zoned support to the patient's body 105 may provide a more uniform overall support to the patient's body 105 over the entire stretcher pad 100 and may reduce pressures acting on the skin surface of the patient's body 105 over the pad 100, thus possibly improving blood flow and minimizing skin tissue break down and/or deformation over the patient's body 105.

FIG. 8 illustrates another exemplary embodiment of a stretcher pad 200. In this embodiment, the stretcher pad 200 includes a protective cover 276 configured to receive the pad 100 within the cover. The cover 276 may be secured over the stretcher pad 200 by, for example, a zipper, hook and loop fastener, etc. The cover may be formed from an elastic nylon material, etc. suitable for at least partly protecting the stretcher pad 200 and providing a generally anti-microbial environment. The cover 276 may also be formed from stretchable poly-urethane, nylon taffeta, etc. within the scope of the present disclosure.

In still another exemplary embodiment, a stretcher pad is configured for use with a stretcher having longitudinal support rails for at least partly securing a patient's body against rolling or sliding off the stretcher pad. The support rails may be mounted to an upper surface of a frame of the stretcher along longitudinal side edges of the frame's upper surface. The support rails may be movable in a generally vertical direction between an extended position and a retracted position. In the extended position the support rails act to at least partly secure the patient's body on the stretcher pad, and in the retracted position the support rails facilitate transfer of the patient's body onto or off of the stretcher pad.

In yet another exemplary embodiment, a stretcher pad may include a pressure management insert having a baffled fluid-retaining chamber. The baffles may be positioned within the chamber to, for example, control fluid movement through the chamber and to substantially uniformly distributing pressure within the chamber.

The stretcher pad is generally disclosed herein for use with a stretcher. However, it is understood that in other exemplary embodiments, a stretcher pad according to the present disclosure may be used, for example, in an ambulance, an operating table, etc.

Numerical dimensions and values are provided herein for illustrative purposes only. The particular dimensions and values provided are not intended to limit the scope of the present disclosure.

Certain terminology is used herein for purposes of reference only, and thus is not intended to be limiting. For example, terms such as “upper,” “lower,” “above,” “below,” “top,” “bottom,” “upward,” “downward,” “upwardly,” and “downwardly” refer to directions in the drawings to which reference is made. Terms such as “front,” “back,” “rear,” “bottom,” and “side,” describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first,” “second,” and other such numerical terms referring to structures do not imply a sequence or order unless clearly indicated by the context.

When introducing elements or features and the exemplary embodiments, the articles “a,” “an,” “the,” and “said” are intended to mean that there are one or more of such elements or features. The terms “comprising,” “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

The description of the disclosure is merely exemplary in nature and, thus, variations that do not depart from the gist of the disclosure are intended to be within the scope of the disclosure. Such variations are not to be regarded as a departure from the spirit and scope of the disclosure.

What is claimed is:

1. A stretcher for use in transporting a body from one location to at least another location in a generally horizontal position and for improving pressure distribution to the body on the stretcher, the stretcher comprising:
a wheeled frame for rolling the stretcher over a surface and transporting the body from one location to at least another location;
a generally elongate pad supported by the wheeled frame for receiving the body in the generally horizontal position on the stretcher; and
a pressure management insert disposed at least partly within the pad for supporting at least part of a trunk of the body on the stretcher.

2. The stretcher of claim 1, wherein the pressure management insert comprises one or more inflatable fluid retaining cells.

3. The stretcher of claim 2, wherein the one or more inflatable fluid retaining cells substantially uniformly distribute pressure over the part of the body's trunk on the fluid retaining cells.

4. The stretcher of claim 1, wherein the pad includes an opening formed in an upper surface thereof, the opening being configured to retain the pressure management insert within the pad.

5. The stretcher of claim 1, wherein the pad further comprises a sloped portion adjacent a longitudinal end of the pad for supporting a portion of at least one foot of the body.

6. The stretcher of claim 5, wherein the pad decreases in thickness from about four inches to about two inches longitudinally along said sloped portion.

7. The stretcher of claim 1, wherein the pad further comprises at least one raised side bolster extending generally longitudinally along a side edge margin of the pad for at least partly retaining the body against rolling or sliding off the pad.

8. The stretcher of claim 1, further comprising a protective cover for receiving the pad within the cover.

9. A stretcher for use in transporting a body from one location to at least another location in a generally horizontal position with improved pressure distribution to the body, the stretcher comprising:
a generally elongate foam pad;
a frame for supporting the foam pad;
a pressure management insert disposed at least partly within the foam pad for supporting at least part of a trunk of the body received on the pad; and
wherein a length dimension of the inflatable pressure management insert is less than or equal to about one-half of a length dimension of the pad.

10. The stretcher of claim 9, wherein the length dimension of the pad is less than about eighty inches, and wherein the pad further comprises a width dimension of less than about thirty-six inches and a thickness dimension of less than about five inches.

11. The stretcher of claim 10, wherein the length dimension of the pad is about seventy-five inches, the width dimension of the pad is about twenty-nine inches, and the thickness dimension of the pad is about four inches or less.

12. The stretcher of claim 9, wherein the length dimension of the inflatable pressure management insert is less than about forty inches and the inflatable pressure management insert is located about fifteen inches or more from a longitudinal end of the pad.

13. The stretcher of claim 9, wherein a value of a surface area of the pressure management insert is about one-half or less than a value of a surface area of the pad.

14. A method for transporting a body in a generally horizontal position from one location to at least another location, the method comprising:
positioning the body in a generally horizontal position on a generally elongate foam pad supported by a frame, wherein the positioning includes:
supporting at least part of the body with the pad so that one or more extremities of the body are on the pad; and
supporting at least part of a trunk of the body with a pressure management insert within the pad, whereby the pressure management insert substantially uniformly distributes pressure along said at least part of the trunk of the body

15. The method of claim 14, wherein the pressure management insert includes a fluid retaining cell.

16. The method of claim 15, wherein the fluid retaining cell is inflatable, and wherein the method further comprises inflating the fluid retaining cell with air.

17. The method of claim 16, comprising inflating the fluid retaining cell with air prior to positioning the body on the foam pad.

18. The method of claim 17, further comprising securing the body on the pad for retaining the body on the pad against rolling or sliding off the pad.

19. The method of claim 16, further comprising moving the body from an emergency transport vehicle to a medical care facility.

20. The method of claim 19, wherein the body remains on the foam pad for twenty-four hours or less.
